(19) United States
(12) Patent Application Publication
(10) Pub. No.: US 2007/0191605 A1
(43) Pub. Date: Aug. 16, 2007

(54) Method for the production of ling-chained quaternary ammonium oxalates and ammonium hydrogenoxalates

(76) Inventor: Klaus Raab, Burgkirchen (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 11/596,172

(22) PCT Filed: Apr. 28, 2005
(86) PCT No.: PCT/EP05/04535

§ 371 Date: Nov. 10, 2006

(30) Foreign Application Priority Data
May 12, 2004 (DE) 10 2004 023 417.5

(51) Int. Cl. C07D 295/02

(52) U.S. Cl. 544/107

(57) ABSTRACT

The present invention relates to a novel method for the production of long-chained quaternary ammonium oxalates and of long-chained quaternary ammonium hydrogen oxalates by reacting amines with dimethyl carbonate and further reacting the intermediate product with oxalic acid or oxalic acid dihydrate.

The present invention relates to a novel method for the production of long-chained quaternary ammonium oxalates and of long-chained quaternary ammonium hydrogen oxalates by reacting amines with dimethyl carbonate and further reacting the intermediate product with oxalic acid or oxalic acid dihydrate.

JP-A-2002 179 614 discloses a method for the production of quaternary ammonium salts with carboxylates as counterions from quaternary ammonium halides and the alkali metal salts of C₁- to C₆-carboxylic acids in water/alcohol mixtures with subsequent extraction. Here, a residual content of halide in the end product cannot be avoided.

Quaternary ammonium oxalates of the formula
[NR₁R₂R₃R₄]₂⊕C₂O₄²⊖
or quaternary ammonium hydrogen oxalates of the formula
[NR₁R₂R₃R₄]⊕HC₂O₄⊖
in which R₁, R₂, R₃, R₄ are alkyl groups can be produced from amines and an alkylating agent such as, for example, alkyl chloride, alkyl bromide or alkyl iodide, and subsequent exchange of the anions chloride, bromide or iodide for oxalate or hydrogen oxalate, for example with the help of an anion exchanger column charged with oxalate or hydrogen oxalate. Since this anion exchange is an equilibrium process, complete exchange of the anions chloride, bromide or iodide for oxalate or hydrogen oxalate can only be achieved with very great difficulty. 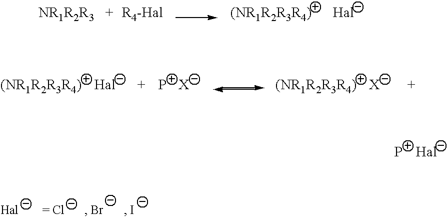
P⊕=polymeric anion exchanger
X⊖=½C₂O₄²⊖ or HC₂O₄⊖

The anion exchanger has to be regenerated again with oxalate solution after the ion exchange process. Since quaternary ammonium compounds with long alkyl groups readily adsorb to the nitrogen atom on various surfaces, including on the large internal surface of ion exchange resins, large amounts of solvent are, moreover, required in order to completely elute the quaternary ammonium oxalates or ammonium hydrogen oxalates.

A further synthesis option for quaternary ammonium oxalates and ammonium hydrogen oxalates is the neutralization of quaternary ammonium hydroxides with the corresponding amount of oxalic acid H₂C₂O₄.
2[NR₁R₂R₃R₄]⊕OH⊖+H₂C₂O₄→[NR₁R₂R₃R₄]₂⊕C₂O₄²⊖+2H₂O
[NR₁R₂R₃R₄]⊕OH⊖+H₂C₂O₄→[NR₁R₂R₃R₄]⊕HC₂O₄⊖+H₂O

The quaternary ammonium hydroxides can be produced from quaternary ammonium halides by anion exchange, for example with the help of an anion exchange column charged with hydroxide. 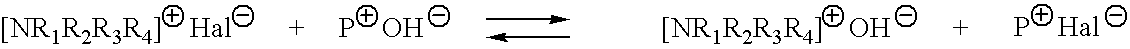
P⊕=polymeric anion exchanger
Hal⊖=Cl⊖, Br⊖, I⊖

The disadvantages described above, such as incomplete anion exchange, adsorption of the long-chained quaternary ammonium salts and consumption of a large amount of solvent, also arise here.

EP 0 251 577 B1 claims quaternary ammonium salts with specific anions as electrolytes for an aluminum electrolyte condenser. Their synthesis takes place by neutralizing short-chained quaternary ammonium hydroxides with the corresponding acids, such as, for example, boric acid, phosphonic acids, silicic acid.

Another synthesis path for quaternary ammonium compounds described in the literature is the reaction of amines with dialkyl carbonates to give quaternary ammonium alkyl carbonates and their further reaction with acids HY.

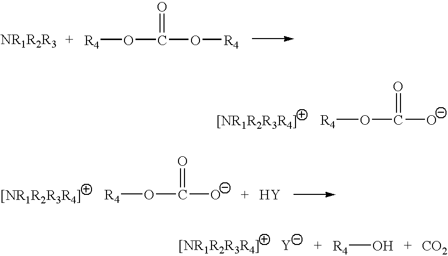

German patent application B 24673 IV c/12 q (1953) describes the reaction of tertiary amines with esters of carbonic acid, such as, for example, dimethyl carbonate, the isolation of the quaternary ammonium salts of carbonic acid and their further reaction with acids, such as, for example, tartaric acid.

IT 1153530 discloses the production of quaternary ammonium alkyl carbonates from amines and dialkyl carbonates and their further reaction with organic or inorganic acids.

The patent application EP 0 291 074 A2 describes the reaction of tertiary amines or phosphines with dialkyl carbonates to give quaternary alkyl carbonates, which are then reacted with a large number of different inorganic or organic acids to give various quaternary ammonium salts or quaternary phosphonium salts.

EP 0 227 179 B1 discloses the use of quaternary ammonium alkyl carbonates or quaternary ammonium benzyl carbonates as corrosion inhibitors, and their production by reacting secondary or tertiary amines with dialkyl carbonates or dibenzyl carbonate.

EP-A-0 345 475 discloses a method for the production of quaternary ammonium salts of the type [N(CH₃)R₁R₂R₃]⊕RCOO⊖, where R is an aliphatic hydrocarbon radical having 8 to 40 carbon atoms, from tertiary amines and dimethyl carbonate or methylethyl carbonate and further reaction with a long-chained aliphatic carboxylic acid RCOOH.

WO 02/00599 A1 and WO 02/00600 A1 disclose quaternary ammonium salts and quaternary phosphonium salts as essential constituents of a novel formulation and their use as agents for stabilizing and isolating nucleic acids from microorganisms. Preferred anions of these quaternary ammonium salts are bromide, chloride, phosphate, sulfate, formate, acetate, propionate, oxalate, malonate, succinate or citrate. The production of quaternary ammonium oxalates is not described.

On account of the described disadvantages of the anion exchange methods, such as, for example, when using anion exchangers, the object underlying the present invention was to develop an improved production method for long-chained quaternary ammonium oxalates and of long-chained quaternary ammonium hydrogen oxalates.

Surprisingly, it has been found that this object can be achieved with a method in which amines are reacted with dimethyl carbonate, and the intermediate product, which may be isolated, but which does not have to be isolated, is further reacted with oxalic acid or oxalic acid dihydrate to give quaternary ammonium oxalates or quaternary ammonium hydrogen oxalates.

The invention therefore provides a method for the production of quaternary ammonium oxalates or of quaternary ammonium hydrogen oxalates of formula (1) 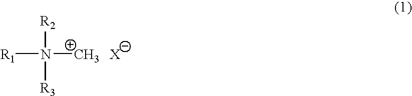
by reacting amines of formula (2) 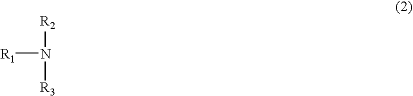
with dimethyl carbonate
and further reacting the intermediate product with oxalic acid or oxalic acid dihydrate, in which
   X⊖ is ½C₂O₄²⊖ or HC₂O₄⊖,
   R₁ is a straight-chained or branched, aliphatic hydrocarbon radical having 8 to 22 carbon atoms, which is saturated or can contain one, two or three C═C-double bonds,
   R₂ is CH₃, C₂H₅, C₃H₇, C₄H₉ or the meaning given for R₁, and
   R₃ is H, CH₃, C₂H₅, C₃H₇, C₄H₉,
      where the reaction of the amine of formula (2) with dimethyl carbonate is carried out under the pressure which is established here in the closed reaction vessel, and the further reaction of the isolated or nonisolated intermediate product with oxalic acid or oxalic acid dihydrate is carried out in a solvent.

R₁ and R₂ are preferably an alkyl radical of the formula CₙH₂ₙ₊₁ where n=8 to 14, where R₁ and R₂ may be identical or different, and R₃ is CH₃ or H. Particularly preferably, R₁ is an alkyl radical of the formula CₙH₂ₙ₊₁ where n=8 to 22, where R₂ and R₃ are then both CH₃. Very particularly preferably, R₁ is C₁₄H₂₉ and R₂ and R₃ are both CH₃.

The reaction of the amine of formula (2) with dimethyl carbonate can be carried out without the addition of a further solvent or in excess dimethyl carbonate as solvent or in an alcohol as solvent, preferably in a short-chained alcohol having 1 to 4 carbon atoms or particularly preferably in methanol as solvent. The mass ratio of solvent to the sum of the masses of amine of formula (2) and dimethyl carbonate can expediently be between 0:1 to 3:1 and, in the case of methanol as solvent, preferably between 0:1 to 2:1, particularly preferably between 0.2:1 to 1:1. Dimethyl carbonate generally only acts as solvent at elevated temperatures. In the case of the reaction of tertiary amines of formula (2) where R₃≠H, the molar ratio of tertiary amine to dimethyl carbonate is preferably between 1:1 and 1:2, particularly preferably between 1:1.2 to 1:1.7. In the case of secondary amines of formula (2) where R₃=H, the molar ratio of secondary amine to dimethyl carbonate is preferably between 1:2 to 1:4, particularly preferably between 1:2.5 to 1:3.5.

The reaction temperature of the reaction of amines of formula (2) with dimethyl carbonate is generally between 100 and 180° C., preferably between 120 and 160° C. The reactor contents are thoroughly mixed, for example by stirring, under the autogenous pressure which is established. The required reaction time can be ascertained through analytical determination of the still unreacted amine, for example by titration.

The intermediate product from the reaction of amines of formula (2) with dimethyl carbonate can also be reacted with oxalic acid or oxalic acid dihydrate without isolation, without further purification or without separating off the solvent. Here, solid oxalic acid, solid oxalic acid dihydrate or oxalic acid or oxalic acid dihydrate dissolved in a solvent to give a solution of the intermediate product in alcohol, preferably in methanol, can be metered in with mixing. A preferred solvent for oxalic acid or oxalic acid dihydrate is water. It is also possible to initially introduce oxalic acid or oxalic acid dihydrate, for example dissolved in water, and to add the intermediate product, still hot in dimethyl carbonate or cooled to room temperature and dissolved in alcohol. The molar ratio of amine of formula (2) to oxalic acid or oxalic acid dihydrate will generally be chosen to be between 0.9:1 and 2.1:1 depending on the target product or desired ratio of oxalate to hydrogen oxalate. To produce long-chained quaternary ammonium oxalates of formula (1) X⊖=½C₂O₄²⊖, the molar ratio of amine of formula (2) to oxalic acid or oxalic acid dihydrate will be between 1.8:1 and 2.1:1, and to produce long-chained quaternary ammonium hydrogen oxalates of formula (1) where X⊖=HC₂O₄⊖, between 0.9:1 and 1.1:1.

The reaction temperature for reacting the intermediate product is advantageously chosen to be between 10 to 80° C., preferably between 20 to 60° C. In order to suppress foam formation as far as possible, mixing should not be too vigorous and the continuous metered addition of one or both reaction components should not be carried out too rapidly. The reaction pressure is not critical. The reaction can expediently take place at atmospheric pressure with withdrawal of reaction gases.

If it is desirable to separate off methanol, excess dimethyl carbonate and the solvents used, such as the alcohols, or some of the water from the compounds of formula (1) according to the invention, then this can be carried out by distillation. The distillation can take place under reduced pressure, at atmospheric pressure or under pressure. Depending on the desired concentrations of the compounds of formula (1) according to the invention, their phase behavior and on the desired, possibly very low concentrations of methanol, dimethyl carbonate or alcohols in the end product, it may be advantageous to add water continuously or discontinuously to the distillation bottom. Since the compounds of formula (1) according to the invention usually foam in water and can form gel phases depending on the concentration and on the solvent, during a distillation, the use of a single-stage or multistage thin-film evaporator is preferred, with distillation being particularly preferably carried out at atmospheric pressure or under reduced pressure. A further advantage of the thin-film evaporator is the only short-term thermal stress of the products.

The invention is now illustrated in more detail using examples.

### EXAMPLE 1

A clear solution of 983 g (4.0 mol) of tetradecyldimethylamine, 540 g (6.0 mol) of dimethyl carbonate and 659 g of methanol were poured, at room temperature, into a cleaned, water-free 4.5 liter stainless steel shaker autoclave. After testing for tightness, the contents were heated slowly to an internal temperature of 140° C. under an N₂ protective-gas atmosphere and with shaking. The autoclave contents were shaken for 4 hours at 140° C. The internal pressure increased to about 20 bar. After cooling to room temperature, the autoclave was carefully decompressed and emptied. Weight of product; 2122 g.

Some (2000 g) of the pale yellow, clear autoclave contents were initially introduced into a 6 liter glass round-bottomed flask fitted with paddle stirrer, internal thermometer, condenser and dropping funnel with pressure compensation. A solution of 236.6 g (1.88 mol) of oxalic acid dihydrate in 2632 g of completely demineralized water was added dropwise with stirring over 3 hours at 25-30° C. and atmospheric pressure. During the dropwise addition, a small amount of foam formed. The liberated gases were removed via the condenser. Following the dropwise addition of the oxalic acid solution, the mixture was stirred for a further 2 hours at 25° C. Weight of product: 4688 g.

Some (1000 g) of the pale yellow, clear solution was dripped into a thin-film evaporator at atmospheric pressure and at temperatures between about 80 and 90° C. to distill off methanol, dimethyl carbonate and a small fraction of the water. Some completely demineralized water was added to the discharged bottom product in order to make up the loss of solvent. The bottom product was dripped again, as described above, into a thin-film evaporator at temperatures from 95 to 105° C. in order to distill off the residual amounts of methanol and dimethyl carbonate. If required, the thin-film evaporator distillation and addition of water can be repeated again. The end product was a 30% strength solution of tetradecyltrimethylammonium oxalate in water free from halide anions.

### EXAMPLE 2

In a 1 liter glass stirred autoclave, after checking for tightness, 123 g (0.50 mol) of tetradecyldimethylamine and 150 g (1.67 mol) of dimethyl carbonate were stirred under an N₂ protective-gas atmosphere for 8 hours at 130° C. and an autogenous pressure of 3-4 bar (absolute). At temperatures above 110° C., the autoclave contents remained liquid, below 110° C. they slowly became solid. The autoclave contents were poured into a 2 liter glass round-bottomed flask fitted with paddle stirrer, internal thermometer, condenser and dropping funnel with pressure compensation, and dissolved in methanol. A concentrated solution of 63 g (0.50 mol) of oxalic acid dihydrate in distilled water was added dropwise at room temperature with slow stirring such that the amount of foam did not increase too considerably. The released gases were removed via the condenser. Following the dropwise addition of the oxalic acid solution, the flask contents were stirred for another half an hour at 50° C.

The clear, pale yellow solution was distilled with stirring using a close-clearance stirrer at 60 to 70° C. and a pressure of initially 900 mbar, which was slowly reduced to about 100 mbar. Excessive concentration and overheating of the flask wall must be avoided since this can lead to undesired gel formation. Following the distillation, a 35% strength solution of tetradecyltrimethylammonium hydrogen oxalate in water was obtained in quantitative yield.

### EXAMPLE 3

In a 2 liter stainless steel stirred autoclave, after checking for tightness, 230 g (1.0 mol) of cocoalkyldimethylamine (cocoalkyl: C₈H₁₇ to C₁₈H₃₇), 126 g (1.4 mol) of dimethyl carbonate and 400 g of isopropanol were stirred under an N₂ protective-gas atmosphere for 8 hours at 140° C. under autogenous pressure. After cooling and decompression, the autoclave contents were poured into a 4 liter glass round-bottomed flask equipped with paddle stirrer, internal thermometer, condenser and dropping funnel with pressure equilibrium. A solution of 64 g (0.51 mol) of oxalic acid dihydrate in completely demineralized water was slowly added dropwise at 40° C. with stirring and the released gases were removed via the condenser. The clear, pale yellow solution was concentrated at 60° C. and a pressure of about 200 mbar in a thin-film evaporator. After two passes, the thin-film evaporator discharge obtained was a 41% strength solution of cocoalkyltrimethylammonium oxalate in water and isopropanol.

1. A method for the production of quaternary ammonium oxalates or of quaternary ammonium hydrogen oxalates of formula (1) 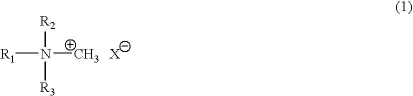
by reacting amines of formula (2) 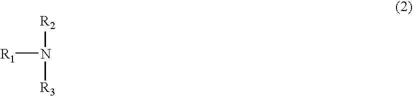
with dimethyl carbonate
and further reacting the intermediate product with oxalic acid or oxalic acid dihydrate, in which
X⊖ is ½C₂O₄²⊖ or HC₂O₄⊖,
R₁ is a straight-chained or branched, aliphatic hydrocarbon radical having 8 to 22 carbon atoms, which is saturated or can contain one, two or three C═C-double bonds,
R₂ is CH₃, C₂H₅, C₃H₇, C₄H₉ or the meaning given for R₁, and
R₃ is CH₃, C₂H₅, C₃H₇, C₄H₉,
where the reaction of the amine of formula (2) with dimethyl carbonate is carried out under the pressure which is established here in the closed reaction vessel, and the further reaction of the isolated or nonisolated intermediate product with oxalic acid or oxalic acid dihydrate is carried out in a solvent.

2. The method as claimed in claim 1, in which the reaction of amine of formula (2) with dimethyl carbonate is carried out in an alcohol, as solvent and in a molar ratio of amine of formula (2) to dimethyl carbonate of from 1:1 to 1:4 or in excess dimethyl carbonate without the addition of a further solvent.

3. The method as claimed in claim 1, in which the reaction of amine of formula (2) with dimethyl carbonate is carried out at 100 to 180° C. under the autogenous pressure of the reaction mixture.

4. The method as claimed in claim 1, in which the further reaction of the isolated or nonisolated intermediate product with oxalic acid or oxalic acid dihydrate is carried out in a solvent selected from the group consisting of water, an alcohol having 1 to 4 carbon atoms or mixtures thereof at 10 to 80° C.

5. The method as claimed in claim 1, in which oxalic acid or oxalic acid dihydrate is metered as aqueous solution into the isolated or nonisolated intermediate product obtained from the reaction of amine of formula (2) with dimethyl carbonate sufficiently slowly and continuously that no foam is formed.

6. The method as claimed in claim 1, in which the molar ratio of amine of formula (2) to oxalic acid or oxalic acid dihydrate is between 0.9:1 and 2.1:1.

7. The method as claimed in claim 1, in which the molar ratio of amine of formula (2) to oxalic acid or oxalic acid dihydrate for producing a quaternary ammonium oxalate is between 1.8:1 and 2.1:1 and for producing a quaternary ammonium hydrogen oxalate is between 0.9:1 and 1.1:1.

8. The method as claimed in claim 1, in which the amine of formula (2) is an alkyldimethylamine, where alkyl is CₙH₂ₙ₊₁ where n=8 to 22 or is a dialkylmethylamine, where alkyl is CₙH₂ₙ₊₁ where n=8 to 14, and the alkyl groups may be identical or different.

9. The method as claimed in claim 1, in which the amine of formula (2) is a tetradecyldimethylamine C₁₄H₂₉N(CH₃)₂.

10. The method as claimed in claim 1, in which, following the reaction with oxalic acid or oxalic acid dihydrate, one or more solvents, are distilled off in a single-stage or multistage thin-film evaporator.

11. The method as claimed in claim 2, in which the alcohol is methanol.

12. The method of claim 10, where the one or more solvents comprises methanol
